(19) United States
(12) Patent Application Publication
(10) Pub. No.: US 2026/0063515 A1
(43) Pub. Date: Mar. 5, 2026

(54) SYSTEMS AND METHODS FOR IMPLANT TESTING WITH HUMANOID ROBOTS

(71) Applicant: Howmedica Osteonics, Corp., Mahwah, NJ (US)

(72) Inventor: Kevin ABBRUZZESE, Mahwah, NJ (US)

(73) Assignee: Howmedica Osteonics Corp., Mahwah, NJ (US)

(21) Appl. No.: 19/318,821

(22) Filed: Sep. 4, 2025

Related U.S. Application Data
(60) Provisional application No. 63/691,089, filed on Sep. 5, 2024.

(51) Int. Cl. G01M 99/00; A61F 2/38; A61F 2/46; B25J 9/16

(52) U.S. Cl. CPC G01M 99/008; A61F 2/468; B25J 9/1661; B25J 9/1679; A61F 2/38

(57) ABSTRACT

A method for gathering test data for one or more medical devices is disclosed, the method comprising: programming a humanoid robot to perform a task, wherein the one or more medical devices are coupled to the humanoid robot; and receiving test data associated with the one or more medical devices during a performance of the task by the humanoid robot. The humanoid robot may include a phenotype selection module, and the method may include determining a phenotype of the humanoid robot based on the phenotype selection module. The phenotype selection module may include a soft tissue model, and the humanoid robot includes one or more electromechanical systems, that may be actuated to conform the humanoid robot to the phenotype determined by the phenotype selection module.

### CROSS-REFERENCE TO RELATED APPLICATION(S)

This patent application claims the benefit of priority to U.S. Provisional Application No. 63/691,089, filed on Sep. 5, 2024, the entirety of which is incorporated herein by reference.

### FIELD OF THE DISCLOSURE

This disclosure relates to testing medical devices with humanoid robots. In particular, this disclosure relates to devices and methods for generating and executing field tests on medical devices with humanoid robots and for tuning humanoid robots in order to gather test data to determine medical device design and postoperative activities after one or more medical procedures, such as a joint replacement procedure, among other aspects.

### BACKGROUND OF THE DISCLOSURE

As medical device technology evolves, there is a need to accurately test the use of the medical device prior to application on a human patient. For example, testing a medical implant to determine flaws in the design prior to the use of the device on a human patient is critical to avoid procedure complications and patient injury. In the context of prosthetic implants, there is a need for rigorous implant life cycle testing in situations that are capable of replicating the subtleties of human motion. Current mechanical testing includes simulator based methods that are limited in their ability to accurately and holistically capture human motion. Humanoid robots may substantially replicate human motion in a manner that provides an opportunity to assess more clinically relevant activities of daily living in different environments. Current mechanical testing strategies maintain systematic constraints that prevent testing in situations that optimally mimic human motion and are further unable to test multiple implants.

Improved systems and methods for collecting and analyzing data to assist in implant testing, design, and selection are desired.

### BRIEF SUMMARY OF THE DISCLOSURE

In some aspects, the techniques described herein relate to a method for gathering test data for one or more medical devices, the method including: programming a humanoid robot to perform a task, wherein the one or more medical devices are coupled to the humanoid robot; receiving test data associated with the one or more medical devices during a performance of the task by the humanoid robot.

In some aspects, the techniques described herein relate to a system for gathering test data for one or more medical devices, the system including: a humanoid robot including one or more processors; one or more medical devices coupled to the humanoid robot; and one or more sensors configured to gather test data associated with the one or more medical devices during a performance of a programmed task by the humanoid robot.

In some aspects, the techniques described herein relate to a system including: a programmable humanoid robot; one or more sensors; and one or more implants coupled to the programmable humanoid robot; wherein the programmable humanoid robot includes: at least one processor; and at least one storage including instructions which, when executed by the at least one processor, cause the at least one processor to perform operations including: receive phenotype data from one or more data sources; determine, using a phenotype selection module, a phenotype of the humanoid robot based on the phenotype data; adjust one or more electromechanical systems to conform the humanoid robot to the phenotype determined by the phenotype selection module; program the humanoid robot to perform a task based on a task selection model; receive test data associated with the one or more implants during a performance of the task by the humanoid robot; and causing to display, on one or more display devices; test outputs based on the test data associated with the one or more implants.

### BRIEF DESCRIPTION OF THE DRAWINGS

A more complete appreciation of the subject matter of this disclosure and the various advantages thereof may be realized by reference to the following detailed description, in which reference is made to the following accompanying drawings:

FIG. 1 is an exemplary testing environment for one or more implants, according to aspects of this disclosure.

FIG. 2 is an exploded illustration of a humanoid robot with a plurality of implants, according to aspects of this disclosure.

FIG. 3 is an illustration of an implant installed on a humanoid robot limb, according to aspects of this disclosure.

FIG. 4 illustrates a system for collection, processing, transmission, and storage of implant test data associated with an operative patient, according to aspects of this disclosure.

FIG. 5 is a schematic diagram depicting the processing of preoperative, intraoperative, and postoperative data and outputs of the system of FIG. 4, according to aspects of this disclosure.

FIG. 6 is a schematic diagram exemplifying types of postoperative data and outputs of the system of FIG. 4, according to aspects of this disclosure.

FIG. 7 illustrates an exemplary testing and simulation diagram for phenotype selection, according to aspects of this disclosure.

FIG. 8 shows an exemplary machine learning training flow chart, according to aspects of this disclosure.

FIG. 9 is a functional block diagram of a computer, according to aspects of this disclosure.

### DETAILED DESCRIPTION

Reference will now be made in detail to the various embodiments of the present disclosure illustrated in the accompanying drawings. Wherever possible, the same or like reference numbers will be used throughout the drawings to refer to the same or like features. It should be noted that the drawings are in simplified form and are not drawn to precise scale. Additionally, the term “a,” as used in the specification, means “at least one.” The terminology includes the words above specifically mentioned, derivatives thereof, and words of similar import. Although at least two variations are described herein, other variations may include aspects described herein combined in any suitable manner having combinations of all or some of the aspects described.

In this disclosure, “user'′ is synonymous with ”practitioner'′ and may be any person completing the described action (e.g., surgeon, technician, nurse, etc.). An implant may be a medical device that is at least partially implanted in a patient and/or provided inside of a patient's body. For example, an implant may be a sensor, artificial bone(s), or other medical device coupled to, implanted in, or at least partially implanted in a bone, skin, tissue, organs, etc. A prosthesis or prosthetic may be a device configured to assist or replace a limb, bone, skin, tissue, etc. Many prostheses are implants, such as a tibial prosthetic component. Some prostheses may be exposed to an exterior of the body and/or may be partially implanted, such as an artificial forearm or leg. Some prostheses may not be considered implants and/or otherwise may be fully exterior to the body, such as a knee brace. Systems and methods disclosed herein may be used in connection with implants, prostheses that are implants, and also prostheses that may not be considered to be “implants” in a strict sense. Therefore, the terms “implant” and “prosthesis” will be used interchangeably and as such, unless otherwise stated, the explicit use of either term is inclusive of the other term. Although the term “implant” is used throughout the disclosure, this term should be inclusive of prostheses which may not necessarily be “implants” in a strict sense. For example, a prostheses may be coupled to a patient at an exterior portion of the patient's body, such as a knee brace or other device coupled to an exterior portion of the patient.

Medical devices discussed in this disclosure may include, for example, implants, prosthetics, or wearable items.

In describing preferred embodiments of the disclosure, reference will be made to directional nomenclature used in describing the human body. It is noted that this nomenclature is used only for convenience and that it is not intended to be limiting with respect to the scope of the invention. For example, as used herein, the term “distal” means toward the human body and/or away from the operator, and the term “proximal” means away from the human body and/or towards the operator. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such system, process, method, article, or apparatus. The term “exemplary” is used in the sense of “example,” rather than “ideal.” Further, relative terms such as, for example, “about,” “substantially,” “approximately,” etc., are used to indicate a possible variation of ±10% in a stated numeric value or range.

Implementation of a humanoid robot in testing implants for any of hip, knee, and/or shoulder prostheses would provide an opportunity to assess multiple joints and laterality in clinically relevant environments. The implementation also allows for integrating multi-axis testing, simulating real-world scenarios, and interactions between different body parts with increased efficiency and reduced costs, and improved repeatability and accuracy. The humanoid robots can mimic human movement patterns, allowing for more accurate testing of implants. In some examples, a humanoid robot may incorporate soft tissue simulations and provide a means to test soft tissue at various joints during human movement patterns. The field data could then be used to improve future simulation methods or develop a predictive algorithm for task based wear, along with optimizing the design of implants and other medical devices, such as, for example, the size, geometry, or material of the devices.

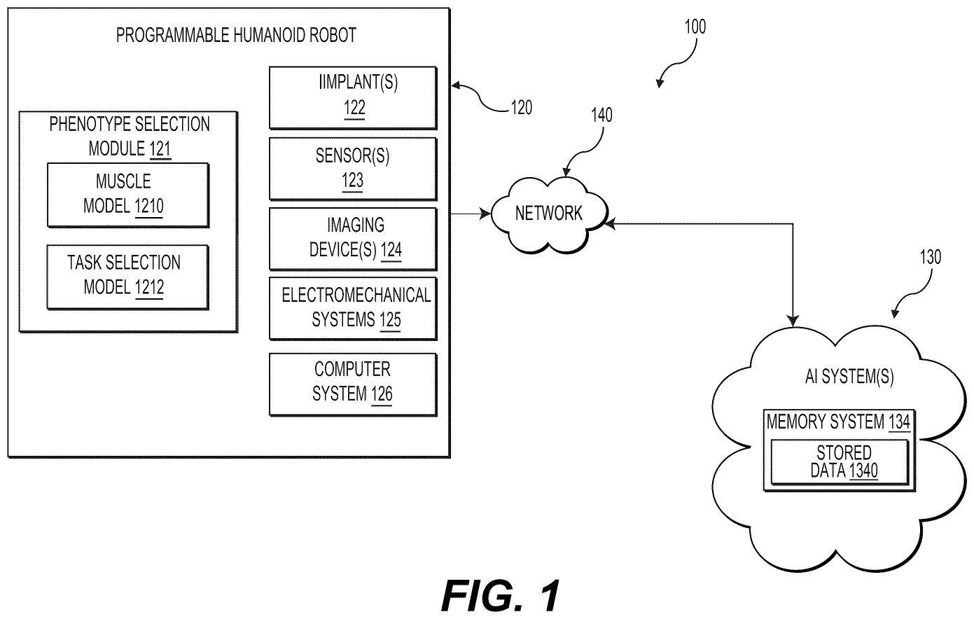

Collecting, storing, processing, and outputting data may occur throughout the course of implant testing and treatment of a patient, including implant testing data, and pre-operative, intra-operative, and post-operative data. Implant testing and data gathering may be performed on a programmable humanoid robot 120, shown diagrammatically in FIG. 1.

FIG. 1 depicts an exemplary environment for testing one or more implants or other medical devices, according to aspects of this disclosure. One or more components of the environment 100 may communicate with one or more of the other components of the environment 100 across electronic network 140, including one or more systems and elements associated with a programmable humanoid robot 120, and one or more systems and elements associated with AI systems 130, which may be stored within a cloud, where cloud may be any local or networked system suitable for transferring data. A central server associated with AI System(s) 130 may be used to manage and synchronize content associated with the humanoid robot 120, ensuring consistent and up-to-date information is transmitted to the humanoid robot 120. The central server may include a central content management system, using a high-level web framework to manage data stored in memory system 134, with content delivery optimized through a content delivery network. Additionally, while a single (e.g., only one) humanoid robot 120 is depicted in FIG. 1, it is understood that environment 100 may include a plurality of humanoid robots 120 (e.g., a network of humanoid robots) without departing from the scope of this disclosure.

The programmable humanoid robot 120 may include a phenotype selection module 121. Phenotype selection module 121 may be used to tune the humanoid robot to a specific patient to test one or more implants according to kinematic and lifestyle information associated with the patient. Phenotype selection module 121 may include at least a muscle model 1210 and a task selection model 1212. In some examples, muscle model 1210 and task selection model 1212 may be incorporated into a single phenotype selection model. Programmable humanoid robot may include one or more implants 122 coupled thereto for purposes of testing the implants or otherwise gathering data related to the implants 122, one or more sensors 123, which may include, for example, inertial measurement units (IMUs), strain gauges, accelerometers, gyroscopes, temperature sensors, etc., one or more imaging devices 124, for example cameras or infrared devices, one or more electromechanical systems 125, which may include, for example, viscoeleastic materials, piezoelectric materials, elastic materials, etc., that may be driven by, controlled by, manipulated by or actuated by motors, hydraulic and/or pneumatic systems, linkages, etc. to mimic or simulate bone, muscle, or other soft tissues, such as, for example, tendons, ligaments, etc., and additional computer systems 126 for operating any of the elements recited.

Muscle model 1210 may be a trained machine learning model stored locally within the phenotype selection module 121, stored in computer system 126, or may be stored in stored data 1340 of a memory system 134 associated with the one or more AI system(s) 130. The term “machine learning model” may generally encompass instructions, data, and/or a model configured to receive input, and apply one or more of a weight, bias, classification, or analysis on the input to generate an output. The output may include, e.g., a classification of the input, an analysis based on the input, a design, process, prediction, or recommendation associated with the input, or any other suitable type of output.

The inputs to muscle model 1210 may include, for example, data related to one or more implants, and data related to one or more patients. For example, the inputs may include information indicating that a patient will undergo a total knee replacement, such that the patient will need a knee implant, and that the patient is a 40-year old female with a body mass index (BMI) of 13.4, a neutral joint line conversion angle (JLCA), and that the patient has indicated that they experience moderate pain associated with their knee. The outputs from the muscle model 1210 may include, for example, instructions for the control of electromechanical systems 125 to optimally tune the programmable humanoid robot to mimic the knee and body of the patient. For example, the knee of the humanoid robot may be adjusted to correlate to the JLCA of the patient and electromechanical actuators as part of electromechanical systems 125 may be adjusted such that the forces experienced at the knee joint correspond to a female patient with a similar BMI of 13.4.

The inputs to the task selection model 1220 may likewise include, for example, data related to one or more implants, and data related to one or more patients. For example, the inputs may include the above information indicating that a patient will undergo a total knee replacement, such that the patient will need a knee implant, and that the patient is a 40-year old female with a body mass index (BMI) of 13.4, a neutral joint line conversion angle (JLCA), and that the patient has indicated that they experience moderate pain associated with their knee. The inputs may also include patient data that indicates tasks that are clinically relevant to the patient. For example, the patient data may include information indicating that the patient is an athlete, in particular a golfer, and that the patient runs 3-5 miles per day. The outputs from the task selection model 1220 may include clinically relevant motions and tasks to be performed by the humanoid robot, including, for example, how much force and torque to apply to the relevant joints or limbs in the completion of those tasks. Furthermore, the outputs from the task selection model 1220 may be inputs into surgical recommendations based on the feedback from the simulated activities to, for example, identify the most appropriate alignments.

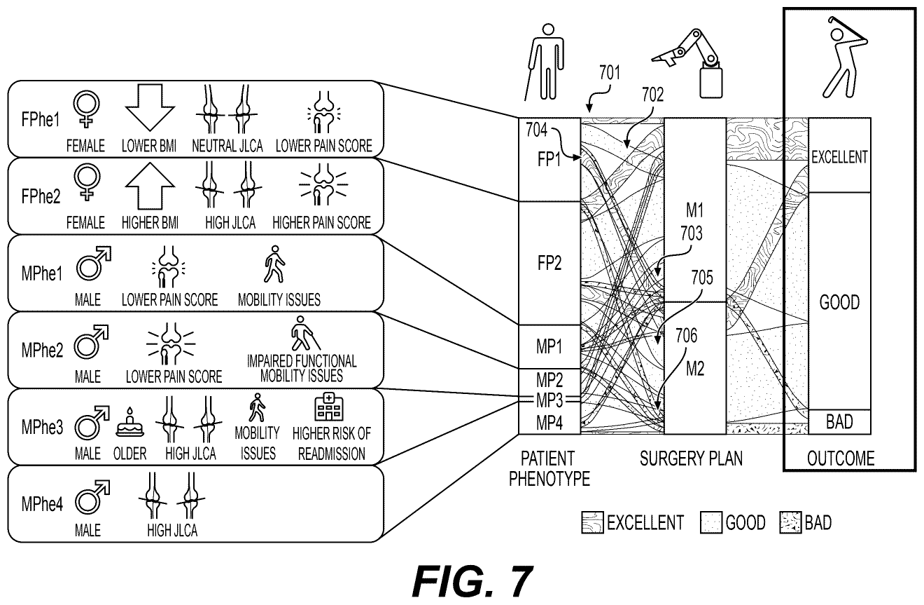

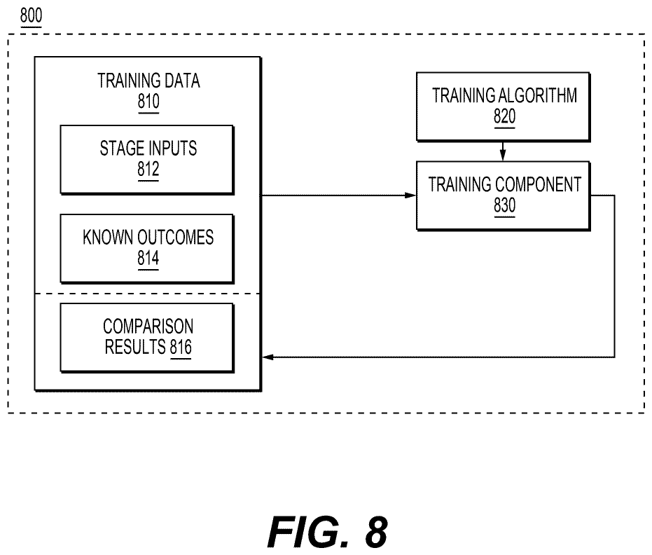

The inputs, outputs, and training of the muscle model 1210 and task selection model 1220 will be discussed in more detail below with regard to FIGS. 7-8. The muscle model 1210 and task selection model 1220 may be continuously learning models as well, where outputs from the models may be used as inputs for future iterations of the model. A machine learning model is generally trained using training data, e.g., experiential data or samples of input data, which are fed into the model in order to establish, tune, or modify one or more aspects of the model, e.g., the weights, biases, criteria for forming classifications or clusters, or the like. The training data may be generated, received, or otherwise obtained from internal or external resources. Aspects of a machine learning system may operate on an input linearly, in parallel, via a network (e.g., a neural network), or via any suitable configuration. By virtue of such training, a machine learning model is converted from an un-trained and un-specific model to a model that is unique to and specifically configured for the particular purpose for which it is trained. In an example, training of a machine learning model is analogous to a method of production in which the article produced is the trained model having unique characteristics by virtue of its particular training. Moreover, the result of training a machine learning model using particular training data and for a particular purpose results in a technical solution to an inherently technical problem.

The execution of the machine learning model may include deployment of one or more machine learning techniques, such as linear regression, logistical regression, random forest, gradient boosted machine (GBM), deep learning, or a deep neural network. Supervised or unsupervised training may be employed. For example, supervised learning may include providing training data and labels corresponding to the training data, e.g., as ground truth. Unsupervised approaches may include clustering, classification, or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc. Alternatively, reinforcement learning may be employed for training. For example, reinforcement learning may include training an agent interacting with an environment to make a decision based on the current state of the environment, receive feedback (e.g., a positive or negative reward based on accuracy of decision), adjusts its decision to maximize the reward, and repeat again until a loss function is optimized.

As discussed above, the humanoid robot may include one or more sensors 123, which may include, for example, IMUs, strain gauges, temperature sensors, gyroscopes, accelerometers, etc. An IMU may include, for example, three gyroscopes and three accelerometers, where a first, second, and third gyroscope and a first, second, and third accelerometer are respectively aligned to three perpendicular axes. Each gyroscope may measure an angular velocity corresponding to a rotation about an axis. In other examples, the IMU may include any number of gyroscopes and any number of accelerometers, may only include one or more gyroscopes and not include accelerometers, or may only include one or more accelerometers and not include gyroscopes.

Each accelerometer may measure a change in motion (acceleration) corresponding to one of the axes. The IMU may include up to nine degrees of freedom (DOF), which may include accelerations, gyroscopic velocities, and magnetometer values for 3-dimensional space. For example, the IMU may include up to 9-DOF, 6-DOF, or 3-DOF, and is not limited to the above-described arrangement. IMU 220 is configured to measure 6 degrees of freedom comprising translation movement along the X axis, Y axis, and Z axis as well as rotational movement such as yaw, roll, and pitch around each axis. Sensors 232 can be added to measure one or more parameters of interest and may differ depending on the application of the device.

The IMU may include one or more micro-electro mechanical (MEMs) integrated circuits. For example, one or more of the gyroscopes or accelerometers may be or include a MEMs integrated circuit. A form factor of a MEMs gyroscope integrated circuit or MEMs accelerometer integrated circuit may support placement in a prosthetic component or coupling to a prosthetic component or bone surface to measure alignment of the muscular-skeletal system. The MEMs gyroscope may have a resonating mass that shifts with angular velocity and output a signal corresponding to (e.g., proportional to) the angular velocity of the IMU. A MEMs accelerometer may have a mass-spring system that shifts in response to an exerted acceleration, e.g., counter to a bias of a spring in the mass-spring system.

The sensors 123 may include other sensors, such as strain gauge sensors, optical sensors, pressure sensors, load cells/sensors, ultrasonic sensors, acoustic sensors, resistive sensors including an electrical transducer to convert a mechanical measurement or response (e.g., displacement) to an electrical signal, etc. Measurement data from the IMU and/or other sensors may be transmitted to a computer system 126 or to AI systems 130 to process and/or display forces, alignment, range of motion, and/or other information from the sensors 123. For example, measurement data from the IMU and/or other sensors may be transmitted wirelessly to a computer or other electronic device to be processed (e.g. via one or more algorithms) and displayed on an electronic display.

Different strains, loads, pressures, forces, etc. measured by each strain gauge sensor and may be processed to determine a load magnitude and location of the load applied. The measured strains and/or other data may be transmitted to the system 20 or another computing platform to calculate load parameters, such as magnitude, location, direction, etc. of an applied load, force, etc. of a joint (e.g., hip joint) in real time, which may then be visualized on a display. Sensors 123 may be incorporated into the implants 122, may be positioned adjacent the implants 122, or may be positioned anywhere on the humanoid robot. In some examples, external sensors may be used for testing with the humanoid robot, such as image sensors or other types of sensors external to the humanoid robot.

One or more imaging devices 124 may also be positioned on the humanoid robot to provide computer vision as an input to the computer systems 126 for control of the humanoid robot 120 and as inputs to the muscle model 1210 and task selection model 1212 in the operation of tasks by the robot. For example, the computer vision provided by the imaging devices 124 may be used for object detection or obstacle detection when performing a given task. The computer vision may also aid in dynamic and adaptive testing, where humanoid robots 120 can perform dynamic movements and adapt to changing conditions, more accurately replicating human behavior and providing more realistic testing conditions.

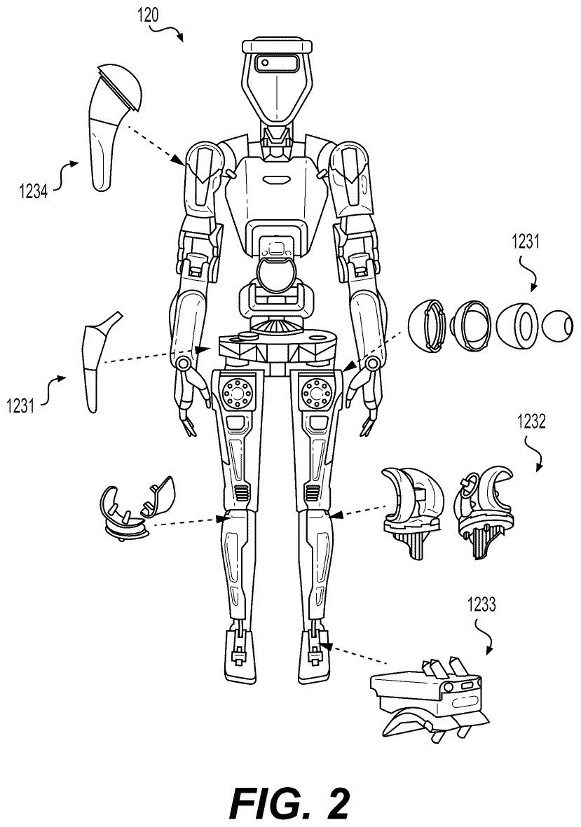

FIG. 2 is an exploded view of a humanoid robot 120 with a plurality of implants 123, according to aspects of this disclosure. Advantageously, humanoid robot 120 may be configured to test multiple implants 123 simultaneously. Current mechanical testing strategies maintain systematic constraints that prevent testing multiple implants and prevent testing of an implants effect on multiple joints. Implementation of a humanoid with implants for either hip 1231, knee 1232, ankle 1233, and/or shoulder 1234 would provide a novel method to assess multiple joints and laterality in clinically relevant environments. The field data could then be used to improve future simulation methods or develop a predictive algorithm for task based wear across multiple joints. One or more of the implants 123 may be coupled to the humanoid robot, with an example of a coupling discussed below. The humanoid robot may collect information related to a plurality of joints when testing a single implant, such as a knee implants effect on hip joints and/or ankle joints, etc.

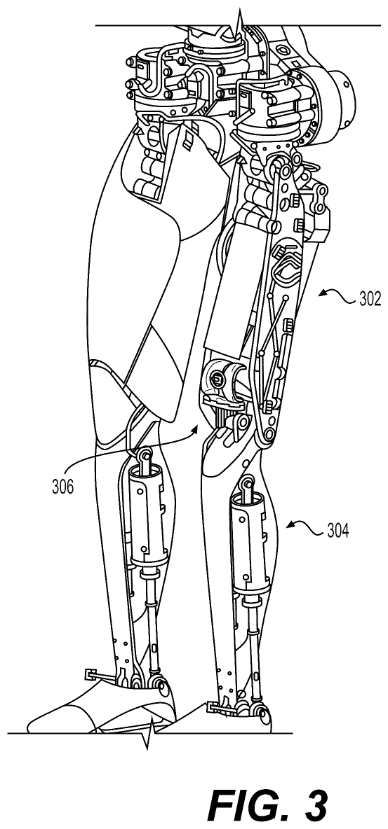

FIG. 3 is an illustration of an implant installed on a humanoid robot. In particular, FIG. 3 depicts a robotic leg 300 of a humanoid robot with a knee implant 306 disposed between an electromechanical upper leg system 302 and an electromechanical lower leg system 304. The electromechanical systems 125 such as the upper leg system 302 and the lower leg system 304 may include actuators, motors (e.g., servo, linear, DC), hydraulic and/or pneumatic systems, linkages, etc. that may be manipulated or designed to mimic or simulate bone, muscle, or other soft tissues, such as, for example, tendons, ligaments, etc., that may be tuned by the phenotype selection module 121 to simulate specific human phenotypes. Human phenotypes will be discussed in more detail with reference to FIG. 7 below.

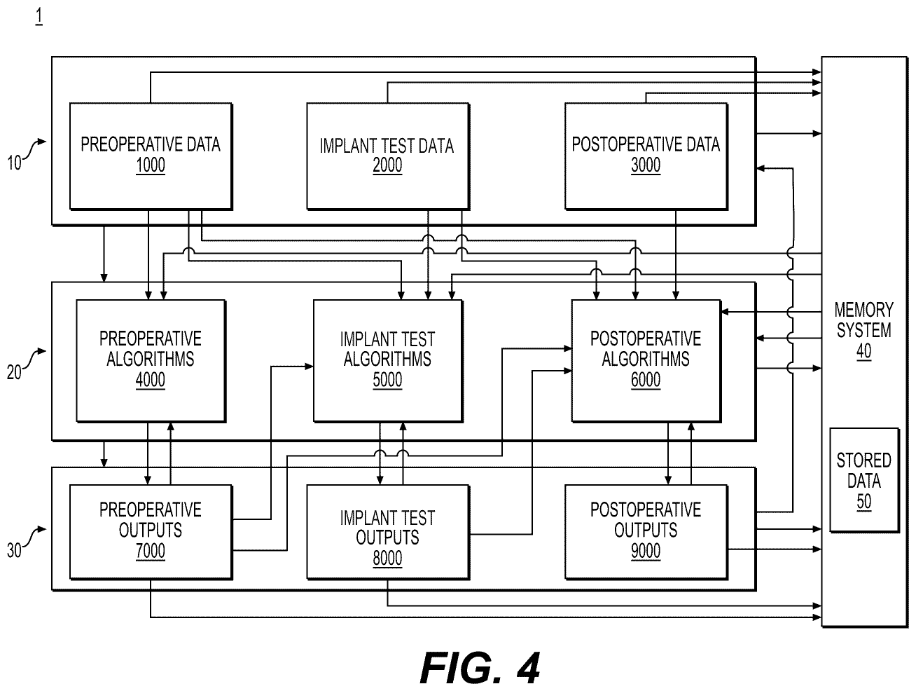

FIG. 4 illustrates an electronic data processing system 1 for collecting, storing, processing, and outputting data throughout the course of implant testing and treatment of a patient, and may be incorporated into any of the measurement systems discussed herein.

Referring to FIG. 4, input information 10 may be input into a system or module 20 to generate output information 30, which may be fed back into system 20 as input information 10. System 20 may be an artificial intelligence (AI) and/or machine learning system. System 20 may include an AI module of AI system 130 (shown in FIG. 1), which may include or communicate with a memory system 40 configured to store the plurality of inputs or input information 10, outputs or output information 30, and stored data 50 from prior patients and/or prior procedures. The input information 10 and output information 30 of an instant procedure may also become stored data 50 and/or used as input information 10 into system 20 and/or memory system 40. Although certain information is described in this specification as being input information 10 or output information 30, due to the continuous feedback loops of data (which may be anchored by memory system 40), the input information 10 described herein may alternatively be determinations or output information 30, and the output information 30 described herein may also be used as input information 10. For example, some input information 10 may be directly sensed or otherwise received, and other input information 10 may be determined or output based on other input information 10.

The input information 10 may include preoperative data 1000, implant test data 2000, and post-operative data 3000. System 20 may perform a plurality of algorithms, such as preoperative algorithms 4000, implant test algorithms 5000, and postoperative algorithms 6000 to generate the output information 30. The output information 30 may include preoperative outputs 7000, implant test outputs 8000, and postoperative outputs 9000. Some or all of the preoperative outputs 7000, implant test outputs 8000, and postoperative outputs 9000 may include determinations such as guidance for medical procedures, guidance for pre-operative or pre-habilitation treatment plans, guidance for post-operative or recovery plans, etc., as will be described in more detail hereinafter. System 20 may include one or more algorithms or modules configured to aggregate results from multiple preoperative algorithms 4000, implant test algorithms 5000, and/or postoperative algorithms 6000 to compile algorithm determinations for certain outputs (e.g., surgical plans, medical treatment plans, or instructions). As shown by the arrows in FIG. 4, the preoperative outputs 7000, implant test outputs 8000, and postoperative outputs 9000 may become inputs into system 20 and/or memory system 40.

Preoperative data 1000 may be data collected, received, and/or stored prior to an initiation of a medical treatment plan or medical procedure. Implant test data 2000 may be data collected, received, and/or stored during a testing of an implant, for example using the programmable humanoid robot 120. Postoperative data 3000 may be data collected, received, and/or stored after completion of the medical treatment or medical procedure.

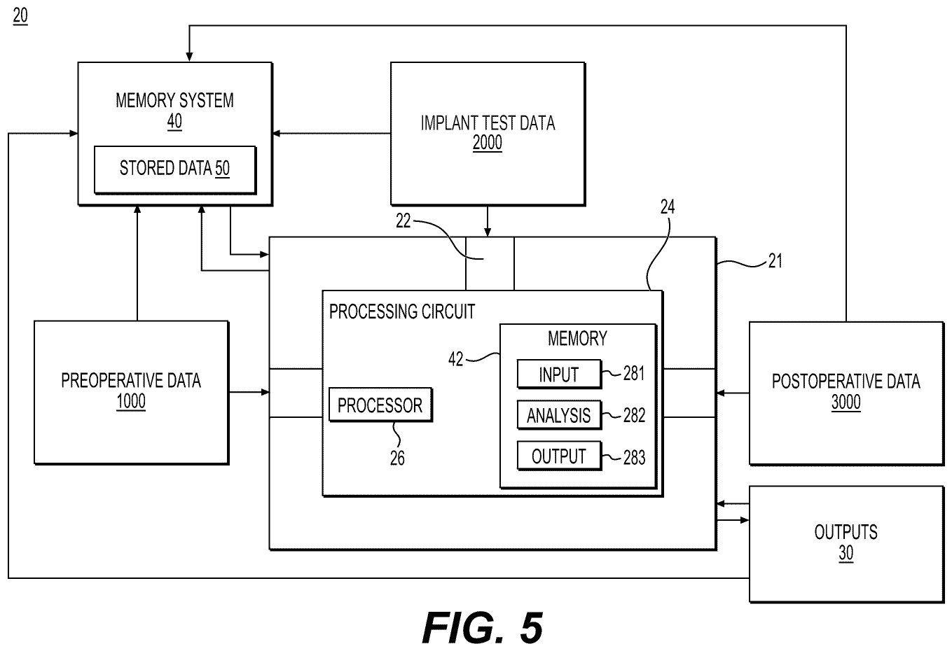

FIG. 5 illustrates an exemplary system architecture for system 20. Referring to FIG. 5, an AI module 21 may be implemented using one or more computing platforms. Examples of one or more computing platforms may include, but are not limited to, smartphones, wearable devices, tablets, laptop computers, desktop computers, Internet of Things (IoT) device, remote server/cloud based computing devices, or other mobile or stationary devices. The AI module 21 may also include one or more hosts or servers connected to a networked environment through wireless or wired connections. Remote platforms may be implemented in or function as base stations (which may also be referred to as Node Bs or evolved Node Bs (eNBs)). Remote platforms may also include web servers, mail servers, application servers, etc.

The AI module 21 may include at least one communication module or interface 22 and a processing circuit 24. The processing circuit 24 may include one or more processors 26 and a memory or storage 42. The memory or storage 42 may be a part of the memory system 40. The memory system 40 is shown in FIG. 2 as providing separate storage from the AI module 21 to exemplify that large amounts of data (e.g., stored data 50) may be stored separately and sent to the AI module 21 via communication modules 22 when needed or where appropriate. However, the memory system 40 may be a part of a computing platform for the AI module 21.

The AI module 21 may be configured to receive the plurality of inputs 10 (the preoperative data 1000, implant test data 2000, and post-operative data 3000), and/or stored data 50 from prior procedures or patients, via the communication module 22. The preoperative data 1000, implant test data 2000, and post-operative data 3000 may be received via manual input or from various sensors. The plurality of inputs 10 may be stored in memory 42 and/or memory system 40. The plurality of input information 10 may be analyzed by processor 24 to determine patterns, such as but not limited to, patterns of movement, wear, force, or displacement. The AI module 21 may be configured to perform the preoperative algorithms 4000, implant test algorithms 5000, and postoperative algorithms 6000 via the processing circuit 24, and to generate the output information 30 via the processor 26.

The communication module 22 may enable wireless communications between the system 20 and the various sensors or data collection devices described herein. The communication module 22 may include wired or wireless interfaces (e.g., jacks, antennas, transmitters, receivers, transceivers, wire terminals, etc.) for conducting data communications with external sources via a direct connection or a network connection (e.g., an Internet connection, a LAN, WAN, or WLAN connection, LTE, 4G, 5G, 6G, Bluetooth, near field communication (NFC), radio frequency identifier (RFID), ultrawideband (UWB), etc.). The communication module 22 may include a radio interface including filters, converters (for example, digital-to-analog converters and the like), mappers, a Fast Fourier Transform (FFT) module, and the like, to generate symbols for a transmission via one or more downlinks and to receive symbols (for example, via an uplink). The communication module 22 may include a BlueTooth module, WiFi module, etc. to receive the input information 10. For example, communication module 22 may include an Ethernet card and port for sending and receiving data via an Ethernet-based communications link or network. In another example, communication module 22 may include a Wi-Fi transceiver for communication via a wireless communications network.

The processing circuit 24 may be configured to implement various functions (e.g., calculations, processes, analyses) described herein. The processor 26 may be implemented as a general purpose processor or computer that may be on-board or in the cloud (e.g. via a remote system, etc.), special purpose computer or processor, microprocessor, digital signal processor (DSPs), an application specific integrated circuit (ASIC), one or more field programmable gate arrays (FPGAs), a group of processing components, processor based on a multi-core processor architecture, or other suitable electronic processing components. The processor 26 may be configured to perform machine readable instructions, which may include one or more modules implemented as one or more functional logic, hardware logic, electronic circuitry, software modules, etc. In some cases, the processor 26 may be remote from one or more of the computing platforms comprising the module 21 and/or system 20. The processor 26 may be configured to perform one or more functions associated with the AI module 21, such as precoding of antenna gain/phase parameters, encoding and decoding of individual bits forming a communication message, formatting of information, and overall control of one or more computing platforms comprising the AI module 21, including processes related to management of communication resources and/or the communication module 22.

The memory 42 may provide an example of the types of devices comprising the memory system 40. The memory 42 may be one or more external or internal devices (random access memory or RAM, read only memory or ROM, Flash-memory, hard disk storage or HDD, solid state devices or SSD, static storage such as a magnetic or optical disk, other types of non-transitory machine or computer readable media, etc.) configured to store data and/or computer readable code and/or instructions that completes, executes, or facilitates various processes or instructions described herein. The memory 42 may be or include volatile memory or non-volatile memory (e.g., semiconductor-based memory device, a magnetic memory device and system, an optical memory device and system, fixed memory, or removable memory). The memory 42 may include database components, object code components, script components, or any other type of information structure to support the various activities described herein. In some aspects or embodiments, the memory 42 may be communicably connected to the processor 26 and may include computer code to execute one or more processes described herein. The memory 42 may contain a variety of modules, each capable of storing data and/or computer code related to specific types of functions. In some embodiments, the memory 42 may contain several modules related to medical procedures, such as an input module 281, an analysis module 282, and an output module 283. The input module 281 may receive input information 10, and the output module 283 may output (e.g., display or transmit) output information 30. The analysis module 282 may include and/or operate the preoperative algorithms 4000, implant test algorithms 5000, and postoperative algorithms 6000.

The AI module 21 and/or system 20 need not be contained in a single housing. Rather, components of the AI module 21 may be located in various different locations or even in a remote location (e.g. remote cloud server system, etc.). Components of the module 21, including components of the processor 26 and the memory 42, may be located, for example, in components of different computers, robotic systems, devices, etc. used in surgical procedures.

The pre-operative data 1000, implant test data 2000, and post-operative data 3000 may be collected using preoperative, implant test, and postoperative measurement systems. The implant test measurement system may include, for example, the programmable humanoid robot 120 and elements of the environment 100 described with reference to FIG. 1. The preoperative algorithms 4000, implant test algorithms 5000, and postoperative algorithms 6000 may be used to generate preoperative outputs 7000, implant test outputs 8000, and postoperative outputs 9000.

Preoperative data 1000 may include any information collected by memory system 40 prior to a medical procedure, such as a surgical procedure or other patient treatment event. The preoperative data 1000 may include information on demographics, lifestyle, medical history, electromyography (EMG), planned procedure, psychosocial information, bone imaging, bone density, biometrics, and kinematics. This list, however, is not exhaustive and preoperative data 1000 may include other patient specific information. Some of the preoperative data 1000 may be directly sensed via one or more devices, may be manually entered by a medical professional, patient, or other party, and other preoperative data 1000 may be determined (e.g., using a preoperative algorithm 4000) based on directly sensed information, input information, and/or stored information from prior medical procedures.

Demographics may include patient age, gender, height, weight, nationality, body mass index (BMI), etc. Lifestyle may include information on smoking habits, exercise habits, drinking habits, eating habits, fitness, thrill-seeking habits and/or risk adverse traits, a type of vehicle a patient drives and movements associated with entering and exiting the vehicle, a type of house or residence the patient lives in and movements associated with climbing and descending stairs, bending movements during daily activities, etc.

Medical history may include allergies, disease progressions, addictions, prior medication use, prior drug use, prior infections, comorbidities, prior surgeries or treatment, prior injuries, prior pregnancies, utilization of orthotics, braces, prosthetics, or other medical devices, etc. EMG information may include information on a muscle response or electrical activity in response to a nerve's stimulation.

Information on a planned procedure may include information about a planned site of the procedure, a disease or infection state, type of procedure to be performed, etc. Alternatively or in addition thereto, a planned procedure may include a surgeon's surgical or other procedure or treatment plan (planned steps or instructions on incisions, bone cuts, implant design, implant alignment, etc.) that was manually prepared by a surgeon and/or previously prepared using one or more algorithms. Psychosocial information may include perceived pain, stress level, anxiety level, mental health status, other feelings and psychosocial data, and other patient reported outcome measures (PROMS). Pyschosocial information may include mental health status and/or information from a Veteran's Rand-12 (VR-12) mental component summary (MCS).

Bone imaging data may include morphology and/or anthropometrics 1082 (e.g., physical dimensions of internal organs, bones, etc.), fractures, slope or angular data, tibial slope, posterior tibial slope or PTS, bone density 1090 (e.g., bone mineral or bone marrow density, bone softness or hardness, or bone impact), etc. Bone density 1090 may be collected separately from bone imaging information 1080 and/or may be collected using, for example, using indent tests or a microindentation tool. Bone imaging data 1080 may not be limited to strictly “bone” and may be inclusive of other internal imaging data, such as of cartilage, soft tissue, or ligaments.

Bone imaging data may include or be used to determine alignment data. Bone imaging data, alignment data, and/or morphology and/or anthropometrics may include data on bone landmarks (e.g., condyle surface, head or epiphysis, neck or metaphysis, body or diaphysis, articular surface, epiconcyle, process, protuberance, tubercle vs tuberosity, trochanter, spine, linea or line, facet, crests and ridges, foramen and fissure, meatus, fossa and fovea, incisure and sulcus, and sinus) and/or bone geometry (e.g., diameters, slopes, angles) and other anatomical geometry data. Such geometry is not limited to overall geometry and may include specific lengths or thicknesses (e. g,, lengths or thicknesses of a tibia or femur). Bone imaging data, alignment data, and/or morphology and/or anthropometrics may also include data on soft tissues for ligament insertions and/or be used to determine ligament insertion sites. For example, bone density may be determined from bone imaging data and may be used to locate or determine a ligament insertion site to balance a knee, or MRI or fMRI data could give additional soft tissue information.

Bone imaging data, alignment data, and/or morphology and/or anthropometrics may include lower extremity mechanical alignment, lower extremity anatomical alignment, femoral articular surface angle, tibial articular surface angle, mechanical axis alignment strategy, anatomical alignment strategy, natural knee alignment strategy, femoral bowing, tibial bowing, patello-femoral alignment, coronal plane deformity, coronal plane deformity that can be passively correctable, sagittal plane deformity, extension motion, flexion motion, anterior cruciate ligament (ACL) ligament intact, posterior cruciate ligament (PCL) ligament intact, knee motion in all three planes during active and passive range of motion in a joint, three dimensional size, proportions and relationships of joint anatomy in both static and motion, height of a joint line, lateral epicondyle, medial epicondyle, lateral femoral metaphyseal flare, medial femoral metaphyseal flare, proximal tibio-fibular joint, tibial tubercle, coronal tibial diameter, femoral interepicondylar diameter, femoral intermetaphyseal diameter, sagittal tibial diameter, posterior femoral condylar offset-medial and lateral, lateral epicondyle to joint line distance, and/or tibial tubercle to joint line distance.

Biometrics may include resting heart rate or heat rate variability, electrocardiogram data, breathing rate, temperature (e.g., internal or skin temperature), skin moisture, oxygenation, sleep patterns (e.g., heart rate variability or HRV, REM cycle data, type of sleep such as REM, deep, or light, sleep frequency, time asleep versus time awake, disturbances in the sleep or periods of movement, patterns in sleep timing or time of day asleep, etc.), and/or activity frequency and intensity. Biometrics may include patient-specific or unique characteristics, such as fingerprint data, DNA or RNA signatures, etc.

Kinematics may include movement or position information at various anatomical areas or locations, muscle function or capability, and range of motion data. Additional kinematics data may include strength measurements and/or force measurements. For example, kinematics may include data used to determine a push-off power, force, or acceleration, or a power, force, or acceleration at a toe during walking. Range of motion data may include a range of motion at one or more joints, such as an angular range or axes of joint motion, or rotation, flexion or extension data. For example, kinematics may include a flexion value, where a flexion value of 180 degrees±3 degrees may indicate a full extension of a joint, and any value other than 180 degrees±3 degrees may indicate a joint in flexion where bones on either side of the joint intersect to form an angle other than 180 degrees. Kinematics may include dynamic information, speed or acceleration information, torque or force information, etc. Some of this information may be estimated or determined based on raw data from motion sensor systems and/or other sensors. For example, kinematics may include how quickly a patient can bend a joint, sit down, stand up, a push-off power during walking, etc. Kinematics may also include steps (e.g., measured by a pedometer) and/or measured gait, including gait metrics such as stride length, stance, cadence, etc. Kinematics may include a number of fall events and/or disoriented events (e.g., measured by an accelerometer, mobile device, etc.) Kinematics may include swaying or other movement which would indicate an unsteady balance of a patient, such as postural sway at the hips, knees, or neck. Kinematics may include pendulum knee drop information. Kinematics may also include and/or indicate frailty, fall risk, and/or joint stiffness (e.g., based on a speed or ease of how a joint is moved through a range of motion).

The kinematics information may include measurements in relation to a leg axis system, such as alignment data or other anatomical measures. Alignment data may be obtained using kinematics information and/or range of motion information, bone imaging data and/or morphology/anthropometrics data, etc. In this way, alignment data may also be a type of preoperative output 7000. Anatomical measures and/or alignment data may include arithmetic hip-knee-ankle angle or aHKA, anatomical hip-knee-ankle angle, medial proximal tibial angle or MPTA, lateral distal femoral angle or LDFA, mechanical axis alignment, anatomic alignment, natural knee alignment, gap balancing, measured resection, etc., and these values may be combined. For instance, a joint line may be a sum of MPTA and LDFA, and a hip-knee-ankle angle or HKA may be a difference between MPTA and LDFA. These values may be used as coordinates on a 2D plane to describe a patient's knee anatomy and/or combined with additional multiplanar data to describe patient knee anatomy in 3D space.

Preoperative data 1000 and/or information stored in the memory system 40 may also include known data and/or data from third parties, such as data from the Knee Society Clinical Rating System (KSS) or data from the Western Ontario and McMaster Universities Osteoarthritis Index (WOMAC).

The system 20 may collect pre-operative data 1000 from electronic devices storing electronic medical records (EMR), patient/user interfaces or applications such as tablets, computers, and cellular phones, diagnostic imaging systems, mobile devices, a motion sensor, pressure sensor, and/or kinesthetic sensing systems, and electromyography or EMG systems. The devices of the preoperative measurement system may each include one or more communication modules (e.g., WiFi modules, BlueTooth modules, etc.) configured to transmit preoperative data 1000 to the memory system 40, the system 20, to each other, etc. The system 20 may use other types of stimulation systems (e.g., configured for a kinematic or EMG response) to collect preoperative data 1000.

The system 20 may collect patient reported data, practitioner assessments, etc. using EMR. For example, EMR may be used to collect data on demographics, medical history, biometrics, and information about a planned procedure. Patient and/or user interfaces may be implemented on mobile applications and/or patient management websites or interfaces such as OrthologIQ®. Patient interfaces may present questionnaires, surveys, or other prompts for patients to enter psychosocial information 1060 such as perceived or evaluated pain, stress level, anxiety level, feelings, and other patient reported outcome measures (PROMS). Practitioners may also report psychosocial information (e.g., qualitative assessments or evaluations) via patient interfaces or other interfaces. Patients may also report lifestyle information via patient interfaces. These patient interfaces may be executed on other devices disclosed herein (e.g., using mobile devices or other computers).

The system 20 may collect imaging information from diagnostic imaging systems, which may include computed tomography (CT) scans, magnetic resonance imaging (MRI), x-rays, radiography, ultrasound, thermography, tactile imaging, elastography, nuclear medicine functional imaging, positron emission tomography (PET), single-photon emission computer tomography (SPECT), etc. The system 20 may use these diagnostic imaging systems to collect bone imaging information, including morphology and/or anthropometrics fractures, and bone density (e.g., bone mineral density or bone marrow density).

Mobile devices may include smartwatches, smartphones, tablets, and other devices known in the art. Mobile devices may execute patient interfaces. In some examples, mobile devices may include sensors and/or applications, which the system 20 may use to collect biometrics and other types of patient specific data. For example, mobile devices (e.g., FitBit, Apple Watch, Hexoskin, Polaris strap, iPhone, etc.) may use cameras, light sensors, barometers, global positioning systems (GPS), accelerometers, temperature sensors (e.g., battery temperature sensors), and/or pressure sensors. In some examples, mobile devices may measure heart rate, electrocardiogram data, breathing rate, temperature, oxygenation, sleep patterns, and also activity frequency and intensity.

The system 20 may use EMG systems to collect EMG data. EMG systems may include one or more electrode attached to skin or muscle to measure electrical activity and/or responses to nerve stimulation. The system 20 may use EMG data to determine nerve damage or disease information. EMG data may include information on muscle activity of various body segments including knee, hip, ankle, tibialis anterior, foot, lower back, shoulder, wrist, elbow, forearm, neck, etc.

The system 20 may use motion sensor and/or kinesthetic sensing systems, which may include motion capture (mocap) systems, external motion sensors, wearable sensors, and/or sensors machine vision (MV) technology. Motion sensor systems may measure motion using an optical or light sensor, an accelerometer, a gyroscope, a magnetometer, a compass, barometer, a global positioning system (GPS), a pressure sensor, etc.

The system 20 may use motion capture systems, which may include markerless motion capture systems and other motion sensors (e.g., wearable sensors) to collect kinematics and range of motion data. External motion sensors may include cameras, optical sensors, infrared sensors, ultrasonic sensors, etc. mounted, for example, in an operating room to monitor motion, heat, etc.

The preoperative outputs 7000 may be determined via one or more preoperative algorithms 4000. The preoperative algorithms 4000 may also consider and/or analyze other previously stored data 50 of memory system 40 to determine preoperative outputs 7000. The preoperative outputs 7000 may include a prehabilitation plan, a procedure, medical treatment, or surgical plan, a postoperative plan, a bone density score, a fall risk or stability score, a morphology score, an EMG score, an activity quality score, a joint stiffness score, a patient readiness score, psychosocial score, a b-score or bone shape score, a push-off power score, and a fracture risk score. This list is not exhaustive, however. A “treatment course” or “course of treatment” may refer to any one of or all of the prehabilitation plan, procedure plan, and postoperative plan and/or their intraoperatively determined and postoperatively determined analogs described later.

The procedure, medical treatment, or surgical plan may include instructions for a surgeon in preparing for and/or performing a procedure (e.g., surgery) on the patient. For example, when the procedure plan is a surgical plan for installation of an implant, the surgical plan may include, for example, a planned number, position, length, slope, angle, orientation, etc. of one or more tissue incisions or bone cuts, a planned type of the implant, a planned design (e.g., shape and material) of the implant, a planned or target position or alignment of the implant, a planned or target fit or tightness of the implant (e.g., based on gaps and/or ligament balance), a desired outcome (e.g., alignment of joints or bones, bone slopes such as tibial slopes, activity levels, or desired values for postoperative outputs 9000), a list of steps for the surgeon to perform, a list of tools that may be used, a planned operating room layout (e.g., positions and/or movement of objects or people in the operating room, such as staff, surgeons, medical or surgical robot, operating room table, patient, cameras, GUI, sensors, or other equipment), etc. The procedure plan may also include predictive or target outcomes and/or parameters, such as target postoperative range of motion and alignment parameters, target fall risk or fracture scores, activity quality scores, and joint stiffness scores. These target parameters may be compared postoperatively to corresponding measured postoperative data 3000 and/or determined postoperative outputs 9000 to determine whether an optimized outcome for a patient was achieved. Furthermore, surgical planning information may be used to achieve a specific phenotype plan where the data is used in a closed feedback loop to inform surgical decisions or assess implants iteratively based on different surgical decisions, re-evaluating device performance with alternative configurations, and/or monitoring the performance of a continued or new prostheses device.

A design of the implant may include, for example, curvatures, shapes, or thicknesses and/or shimming parameters corresponding to a patient's anatomy (e.g., from bone imaging data). For example, a design of the implant and/or prosthetic may be configured to match an arc of curvature of the implant with an arc of curvature of the native femoral condyle of the patient, an arc or curvature of a socket area or acetabulum, an arc or curvature of a glenoid or humerus, an arc or curvature of a tibial condyle, etc. Aspects disclosed herein may be applied to a custom knee implant design, custom hip implant design, custom partial knee or hip implant design, or custom design of any other implant design for any other part of a patient's anatomy. The design of the implant may also include materials of the implant and/or placement of implants of autologous tissue, allograft tissue, and/or synthetic materials. The design of the implant may include thicknesses, a number of shims configured to be stacked and/or removed, a size of an added shim, or other dimensions configured to adjust a fit or tightness of the implant.

The postoperative plan may include plans similar to the prehabilitation plan such as an exercise program configured to decrease a recovery time of the patient. The postoperative plan may further include a medication plan (e.g., pain medication plan including a type, dosage, and/or tapering of pain medication) and/or a discharge plan including a length of stay in a hospital. The postoperative plan may include a schedule of follow-up visits with a practitioner, surgeon, physical therapist, etc. The postoperative plan may also include a plan for revision surgeries or future additional surgeries, though the procedure plan may be configured to reduce a likelihood of revision procedures or surgeries. Like the prehabilitation plan and procedure plan, the postoperative plan may be based on other preoperative outputs 7000. For example, the postoperative plan may include an exercise program configured to target muscles based on the patient's lifestyle (e.g., frequency of climbing stairs or frequency of entering/exiting cars), the fall risk score, and/or the fracture score. The procedure plan may be updated and/or modified based on implant test information 2000 and postoperative information 3000.

The bone density score may be calculated from bone density data, bone imaging data (e.g., morphology/anthropometrics data), medical history, and/or other information input by a patient or practitioner. The bone density score may be implemented as a T-score, defined as the difference between a patient's bone mineral density and 0, which is the bone mineral density of a healthy young adult. where a higher score correlates to a greater bone density, but aspects disclosed herein are not limited.

The fall risk score may be calculated from kinematics, range of motion (e.g., postural sway), and alignment. The fall risk score may be paired with or be calculated based on lifestyle data. For example, the fall risk score may be calculated on a mobile device, be updated based on information sensed by the mobile device, and be displayed on the mobile device (e.g., in a fall risk tracking app). The fall risk score may also be based on other preoperative outputs and/or qualitative observations or scores (e.g., frailty based on walking patterns or walking patterns assessed based on height and/or weight) and/or other observations input by a practitioner or patient (e.g., using EMR and/or interfaces). A higher fall risk score may indicate a higher likelihood that a patient will fall or lose balance, or a higher frailty of the patient, but aspects disclosed herein are not limited.

The joint stiffness score may be calculated based on bone imaging, kinematics (e.g., how quickly a patient can bend a joint), range of motion, alignment, etc. Each joint (e.g., knee, hip, ankle, neck) may have its own joint stiffness score. A higher joint stiffness score may mean a higher stiffness and/or less laxity at the joint, but aspects disclosed herein are not limited.

The push-off power score may be based on kinematics, such as measured force, acceleration, contact pressure, etc. at a foot during walking (e.g., from a sensor in a shoe, coupled to the shoe, or coupled to the leg). A higher push-off power score may indicate a faster or stronger push-off during walking or spring in a step. Alternatively or in addition thereto, the push-off score may be measured at the hands, such as during push-ups.

The fracture risk score may be calculated from kinematics, range of motion (e.g., postural sway), bone density, and alignment. The fracture risk score may be paired with or be calculated based on lifestyle data and/or the fall risk score. The fracture risk score 7140 may also be based on other preoperative outputs 7000 and/or qualitative observations or scores (e.g., frailty based on walking patterns or walking patterns assessed based on height and/or weight) and/or other observations input by a practitioner or patient (e.g., using EMR and/or interfaces). As an example, a lower bone density score and a higher fall risk score may result in a higher determined fracture risk score. A higher fracture risk score may indicate a higher likelihood that a patient will fracture a bone, or a higher frailty of the patient, but aspects disclosed herein are not limited. Any of the scores discussed herein, such as fall risk score or facture risk score, may be determined with data from a humanoid robot configured to mimic one or more attributes of a patient. These attributes of the patient may be a shape of the patient's bones, a poster, a gate, a flexibility, or any other attribute described herein.

The implant test data 2000 may include information on a surgical plan for installation of an implant, implant test kinematics, implant test kinetics, and where the implant test uses the programmable humanoid robot 120, humanoid robot computer vision, humanoid robot activity, and humanoid robot test environment. The surgical plan may include, for example, a planned number, position, length, slope, angle, orientation, etc. of one or more tissue incisions or bone cuts, a planned type of the implant, a planned design (e.g., shape and material) of the implant, a planned or target position or alignment of the implant, a planned or target fit or tightness of the implant (e.g., based on gaps and/or ligament balance), a desired outcome (e.g., alignment of joints or bones, bone slopes such as tibial slopes, activity levels, or desired values for postoperative outputs 9000).

Implant test kinematics may include movement or position information at various areas or locations at or adjacent an implant, and range of motion data. Additional kinematics data may include strength measurements and/or force measurements. For example, kinematics may include data used to determine a push-off power, force, or acceleration, or a power, force, or acceleration at a toe during walking. Range of motion data may include a range of motion at one or more joints, such as an angular range or axes of joint motion, or flexion or extension data. For example, kinematics may include a flexion value, where a flexion value of 180 degrees±3 degrees may indicate a full extension of a joint, and any value other than 180 degrees±3 degrees may indicate a joint in flexion where bones on either side of the joint intersect to form an angle other than 180 degrees. Implant test kinematics may include dynamic information, speed or acceleration information, torque or force information, etc. Some of this information may be estimated or determined based on raw data from motion sensor systems and/or other sensors on the programmable humanoid robot. For example, kinematics may include how quickly the humanoid robot can bend a joint, sit down, stand up, a push-off power during walking, etc. Implant test kinematics may also include steps (e.g., measured by a pedometer) and/or measured gait. Implant test kinematics may include a number of fall events and/or disoriented events (e.g., measured by an accelerometer, mobile device, other one or more sensors, etc.) Implant test kinematics may include swaying or other movement which would indicate an unsteady balance of a patient, such as postural sway at the hips, knees, or neck. Implant test kinematics may include pendulum knee drop information. Implant test kinematics may also include and/or indicate frailty, fall risk, and/or joint stiffness (e.g., based on a speed or ease of how a joint is moved through a range of motion).

The implant test kinematics information may include measurements in relation to a leg axis system, such as alignment data or other anatomical measures. Alignment data may be obtained using implant test kinematics information and/or range of motion information, bone imaging data and/or morphology/anthropometrics data, etc. In this way, alignment data may also be a type of preoperative output 7000. Anatomical measures and/or alignment data may include arithmetic hip-knee-ankle angle or aHKA, anatomical hip-knee-ankle angle, medial proximal tibial angle or MPTA, lateral distal femoral angle or LDFA, mechanical axis alignment, anatomic alignment, natural knee alignment, gap balancing, measured resection, etc., and these values may be combined. For instance, a joint line may be a sum of MPTA and LDFA, and a hip-knee-ankle angle or HKA may be a difference between MPTA and LDFA. These values may be used as coordinates on a 2D plane to describe a patient's knee anatomy.

Implant test kinetics may also include forces experienced on one or more test implants 123. The forces may include active forces experienced by the implant during programmed activity, passive forces experienced at the implant during programmed activity, and contact forces acting on the implant surfaces. The programmed humanoid robot activity and humanoid robot test environment may be determined by system 20 based on the preoperative data 1000, and feedback postoperative data 3000. The humanoid robot computer vision may be used to determine and ensure appropriate test activity and test environment.

The implant test outputs 8000 may be determined via one or more implant test algorithms 5000. The implant test algorithms 5000 may also consider and/or analyze other previously stored data 50 of memory system 40 to determine implant test outputs 8000. The implant test outputs 8000 may include implant recommendations, component placement/alignment, soft tissue performance estimates, such as maximum weight limits and maximum ranges of motion, postoperative activity recommendations (type and duration), and implant design analytics, such as longevity of the implant, durability of the implant, etc.

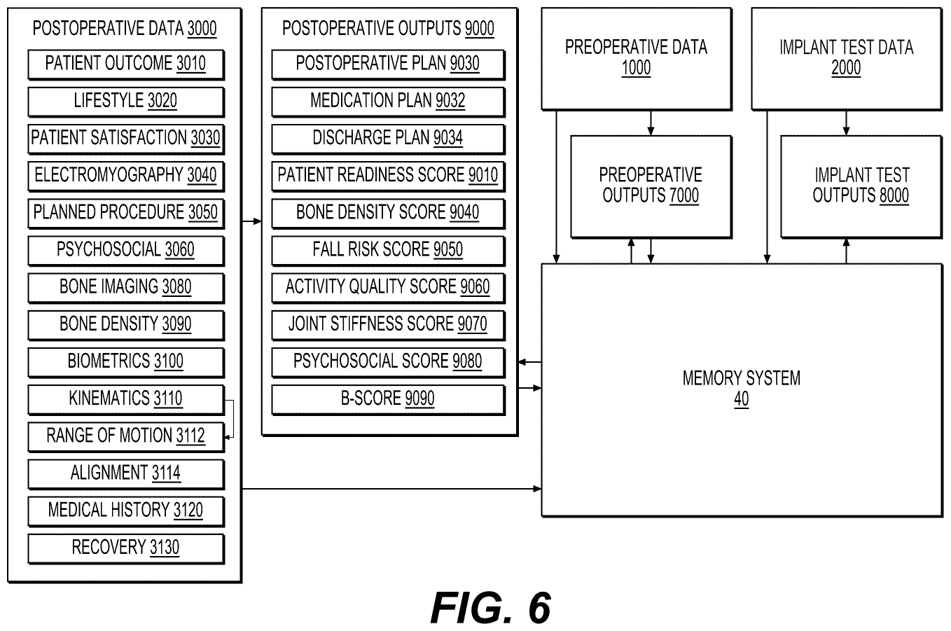

As shown in FIG. 6, implant test data 2000 and implant test outputs 8000 may be incorporated into determining postoperative outcomes. Postoperative data 3000 may include information on patient outcome 3010, lifestyle 3020, patient satisfaction 3030, electromyography (EMG) 3040, planned procedures 3050 (e.g., revisions), psychosocial 3060, bone imaging 3080, bone density 3090, biometrics 3100, and kinematics 3110 including range of motion 3112 and/or alignment 3114, postoperative medical history 3129, and recovery 3130. This list, however, is not exhaustive and postoperative data 3000 may include other patient specific information and/or other inputs manually input by a practitioner. Some of the postoperative data 3000 may be directly sensed, and other postoperative data 3000 may be determined (e.g., using a postoperative algorithm 6000) based on directly sensed or input information.

Patient outcome 3010 may include both immediate and long term results and/or metrics from the medical procedure (e.g., surgery). For example, patient outcome 3010 may include a success metric or an indication of whether the procedure was successful, changes in range of motion, stability, fall risk or stability, fracture risk, joint stiffness or flexibility, or other changes between preoperative data 1000, or intraoperative data 2000 and postoperative data 3000, etc. Patient satisfaction 3030 may be a patient-reported (or, alternatively or in addition thereto, a practitioner-reported) satisfaction with the procedure, both immediate and long-term. Planned procedure 3050 may include information determined in outputting the postoperative plan 8050 and/or other information on future planned procedures for the patient (e.g., a surgeon-created plan or revision based on patient outcome 3010, etc.) Furthermore, humanoid PROMs information may be prompted given the designated phenotype to infer recovery trends or predict PROM information. Medical history 3120 may include updated and/or new medical history 3120 (as compared to preoperative medical history 1030) and may include both immediate and long term information such as new utilization of orthotics, care information in a supervised environment such as a skilled nursing facility or SNF, infection information, etc. Information on recovery 3130 may include information on adherence to a postoperative plan 8030 such as actual exercises performed, medicine dosage and/or type actually taken, fitness information, planned physical therapy (PT), adherence to PT, etc. Information on recovery 3130 may also include discharge and/or length of stay information.

Lifestyle 3020, EMG 3040, psychosocial 3060, bone imaging 3080, bone density 3090, biometrics 3100, kinematics 3110, range of motion 3112, and/or alignment 3114 may include similar types of information as preoperative lifestyle 1020, EMG 1040, psychosocial 1060, bone imaging 1080, bone density 1090, biometrics 1100, kinematics 1100, range of motion 1112, and alignment 1114. For example, psychosocial 3060 may include perceived pain, stress, happiness, anxiety, etc.

The postoperative outputs 9000 may be determined via one or more postoperative algorithms 6000. The postoperative algorithms 6000 may also consider preoperative information 1000 and/or outputs 7000, implant test data 2000 and/or outputs 8000, and/or other previously stored data 50 of memory system 40 to determine postoperative outputs 9000. The postoperative outputs 9000 may include an updated or new postoperative plan 9030, which may include a medication plan 9032 (e.g., for pain medication, antibiotics, etc.) and/or a discharge plan 9034, a patient readiness score 9010, an updated or new bone density score 9040, an updated or new fall risk or stability score 9050, an updated or new activity quality score 9060, an updated or new joint stiffness score 9070, an updated or new psychosocial score 9080, an updated or new B-score 9090, an updated or new push-off power score 9100, and an updated or new fracture risk score 9140. This list is not exhaustive, however. The updated or new bone density score 9040, fall risk or stability score 9050, activity quality score 9060, joint stiffness score 9070, psychosocial score 9080, B-score 9090, push-off power score 9100, or fracture risk score 9140 may include any of the features of preoperatively and intraoperatively determined data. The patient readiness score 9010 may indicate a readiness to be discharged (rather than a readiness for surgery) and may be based on (and updated using) postoperative data and outputs 3000, 9000, such as patient outcome 3010, lifestyle 3020, patient satisfaction 3030, electromyography 3040, psychosocial 3060, bone imaging 3080, biometrics 3100, kinematics 3110, recovery 3130, fall risk score 9050, activity quality score 9060, psychosocial score 9080, push-off power score 9100, fracture risk score 9140, etc.

As previously mentioned, postoperative algorithms 6000 may be used to output the postoperative plan 9030. This postoperative plan 9030 may be newly generated based on postoperative data 3000 and/or may be a modification to the postoperative plan 8030 generated using the intraoperative data 2000 (and/or a manually input) and/or the postoperative plan 7030 generated using the preoperative data 1000 (and/or manually input). In this context, for example, a medical practitioner may manually input an adjustment to the postoperative plan 9030 via an electronic device. The postoperative plan 9030 may be continuously adjusted and/or updated as more postoperative data 3000 is collected.

As an example, the postoperative algorithm 6000 may determine that only minor adjustments are necessary to update the postoperative plan 8030 based on postoperative data 1000 like recover 3130, kinematics 3110, biometrics 3100, patient satisfaction 3030, lifestyle 3020, etc. As another example, unexpected responses or conditions indicated by the postoperative data 1000, which may differ from expected or optimized postoperative conditions (e.g., increased or decreased perceived pain, lower or higher range of motion 3112, unexpected injury indicated in the medical history 3120, etc.), may be analyzed and considered, and the postoperative algorithm 6000 may generate a new postoperative plan 9030 (e.g., based on stored data 50 from other patients with similar unexpected conditions).

The postoperative plan 9030 may include any of the features of the prehabilitation plan 7010 or procedure plans 7020, 8020, and/or 9020, such as an exercise program configured to decrease a recovery time of the patient. The postoperative plan 9030 may include a medication plan 9032 (e.g., pain medication plan including a type, dosage, and/or tapering of pain medication) and/or a discharge plan 9034 including a length of stay in a hospital. The medication plan 9032 may be based on psychosocial information 3060, and may further be based on biometrics 3100 (e.g., heart rate variability and/or sleep patterns).

The postoperative plan 9030 may include any of the features of the preoperatively determined postoperative plan 7030, and may include a schedule of follow-up visits with a practitioner, surgeon, physical therapist, etc. The medication plan 9032 may include instructions for pain medication or other medication (e.g., antibiotics). For example, the medication plan 9032 may include a medication type, active ingredient, mechanism of action, route of administration, dosage level, dosage plan (e.g., taper plan of dosing), frequency, and/or other instructions related to taking medication. The medication plan 9032 may be based on postoperative data 3000 and postoperative outputs 9000. For example, the medication plan 9032 may be based on a patient's prior drug history, perceived pain and/or PROMS, biometrics 3100 like heart rate variability and sleep patterns, bone imaging 3080 (e.g., fractures or healing of fractures), infections or sickness (e.g., detected from changes in synovial fluid using sensored implants, detected from sensors measuring blood glucose, body temperature, sleep disturbances, heart rate variability, etc.) and other recovery 3130 data.

The medication plan 9032 may be updated continuously and/or periodically postoperatively. For example, biometrics 3100 like certain heart rate variability patterns (e.g., higher heart rate) and/or short or infrequent sleeping patterns may indicate that the patient is experiencing a higher level of pain, and the medication plan 9032 may be updated to increase a dose or determine a different (or stronger) type of pain medication. As another example, sensored implants may detect information related to infections, and the medication plan 9032 may be updated to include an antibiotic or other type of medication meant to treat the infection. Infection information may be sensed by sensored implants 316A or other sensors configured to detect a change in synovial fluid and configured to detect other biometrics 3100 such as heart rate variability, blood glucose, sleep disturbances, and body temperature which may indicate an infection at the surgical site. As another example, addictive behaviors may be determined (e.g., using biometrics 3100 and EMG data 3040 in combination with patient medical history or lifestyle 1020 and/or 3020 and/or other PROMS data), and the medication plan 9032 may be created or updated to avoid and/or taper addictive pain medication, like opioids.

The discharge plan 9034 may include instructions for immediate recovery after surgery, such as a length of a hospital stay, supervision instructions, physical therapy instructions, target outputs (e.g., a fall risk threshold or target for the fall risk score 9050, a target activity quality threshold or target for the activity quality score 9060, a target patient readiness score 9010, a push-off power threshold or garget for the push-off power 9100, and/or a fracture risk threshold or target for the fracture risk score 9140), etc. The discharge plan 9034 may be based in preoperative, implant test, and postoperative data and outputs 1000, 2000, 3000, 7000, 8000, an/or 9000. For example, the discharge plan 9032 may be based on recovery 3130, medical history 3120, patient satisfaction 3030, patient outcome 3010, bone imaging 3080, kinematics 3110, biometrics 3100, fall risk score 9050, activity quality score 9060, bone density score 9040, patient readiness score 9010, push-off power 9100, and/or fracture risk score 9140.

For example, the discharge plan 9034 (and/or the patient readiness score 9010) may be updated using and/or based on postoperatively determined fall risk or stability score 9050 and/or postoperatively determined fracture risk score 91. The fall risk or stability score 9050 may be determined and/or updated using kinematics 3110 and biometrics 3100. The fracture risk score 9140 may be determined using fall risk score 9050 (or any inputs used to calculate the fall risk score 9050) and bone density data 3090 and/or a determined bone density score 9040. The fall risk score 9050 and/or fracture risk score 9140 may increase, for example, based on certain (e.g., increased) heart rate combined with exit events and other kinematics data 3110 (e.g., acceleration data) from sensored implants 216A. Based on an increased fall risk score 9050 and/or fracture risk score 9140, the discharge plan 9034 may be updated to increase a number of days in the hospital.

The algorithms 4000, 5000, and 6000 described herein may be further trained and/or refined over time for the instant patient and/or future patients based on all data 1000, 2000, and 3000 and outputs 7000, 8000, and 9000 may be stored in the memory system 40, including patient outcome 3010 data. For example, the algorithms 4000, 5000, and 6000 may learn and/or determine relationships across various data and parameters and make determinations (e.g., for an implant design or tightness, for exercises to include in pre or postoperative exercise plans, for medication plans, length of stay, etc.) based on those new learned, trained, and/or determined relationships.

For an instant patient, the system 20 may use multiple preoperative algorithms 4000, implant test algorithms 5000, and postoperative algorithms 6000 prior to, during, and after a medical procedure to continuously monitor and track the patient and update or refine treatment and implant testing.

FIG. 7 illustrates an exemplary testing and simulation diagram for phenotype selection, as part of the implant test data gathering and test implementation. Phenotype selection is an AI-based implant test algorithm to generate implant test outputs 8000 as described below. Stored data 1340 in memory system of FIG. 1 and/or stored data 50 in memory system 40 of FIG. 5 may be transmitted to system 20 and/or phenotype selection module 121. Phenotype selection may be determined at phenotype selection module 121, or as an implant test algorithm 5000 performed at system 20.

Advantageously, a programmable humanoid robot 120 can be altered to test implants in a variety of environments and varied to a plurality of patients. The different types of patients are referred to as different phenotypes, and the phenotype selection module 121 can tune the humanoid robot 120 to a particular phenotype based on the patient. The phenotype selection module 121 may receive input information 10 to determine a phenotype selection.

As discussed above with reference to FIG. 4, the input information 10 may include preoperative data 1000, implant test data 2000, and post-operative data 3000. System 20 may perform a plurality of algorithms, such as preoperative algorithms 4000, implant test algorithms 5000, and postoperative algorithms 6000 to generate the output information 30. The output information 30 may include preoperative outputs 7000, implant test outputs 8000, and postoperative outputs 9000. Some or all of the preoperative outputs 7000, implant test outputs 8000, and postoperative outputs 9000 may include determinations such as guidance for medical procedures, guidance for pre-operative or pre-habilitation treatment plans, guidance for post-operative or recovery plans, etc., as will be described in more detail hereinafter. System 20 may include one or more algorithms or modules configured to aggregate results from multiple preoperative algorithms 4000, implant test algorithms 5000, and/or postoperative algorithms 6000 to compile algorithm determinations for certain outputs (e.g., surgical plans, medical treatment plans, or instructions). As shown by the arrows in FIG. 4, the preoperative outputs 7000, implant test outputs 8000, and postoperative outputs 9000 may become inputs into system 20 and/or memory system 40.

For example, from EMR data from preoperative data 1000, a patient's gender, age, body mass index (BMI), and joint line convergence angle (JLCA) may be determined. From the psychosocial scores and preoperative outputs 7000, a patient's pain scores, mobility issues, and risk of readmission, for example, may be determined. Other data and outputs described above may also be used in phenotype selection. In the example shown in FIG. 7, six phenotypes are shown: FP1, FP2, MP1, MP2, MP3, and MP4. Phenotype FP1 corresponds to a female patient with a lower BMI, a neutral JLCA, and a lower pain score. Based on a series of test simulations, different surgery plans may be suggested, and different postoperative outcomes tracked. For example, in some cases, a first surgery plan M1 is suggested for the patient with phenotype FP1. In other cases, a second surgery plan M2 is suggested for the patient with phenotype FP1. M1 and M2 may differ in terms of, for example, alignment, component type, component placement, etc. Furthermore, the subset of those surgeries that resulted in “excellent,” “good,” or ““bad” postoperative outcomes are tracked and recorded as part of postoperative data 8000.

In the example shown in FIG. 7, the subset 701 of FP1 cases resulted in an excellent postoperative outcome after an M1 surgery, the subset 702 resulted in a good postoperative outcome after an M1 surgery, and the subset 703 resulted in a bad outcome after an M1 surgery. Furthermore, the subset 704 of FP1 cases resulted in an excellent postoperative outcome after an M2 surgery, the subset 705 resulted in a good postoperative outcome after an M2 surgery, and the subset 706 resulted in a bad outcome after an M2 surgery. Similar postoperative outcomes are measured for the remaining phenotypes, and the postoperative outcomes may be included in the postoperative data 8000 that may be used to further tune the phenotype selection module 121 for implant testing and data gathering. FIG. 8 shows an example machine learning training flow chart 800, according to some embodiments of the disclosure.

Referring to FIG. 8, a given machine learning model is trained using the training flow chart 800. The training data 810 includes one or more of stage inputs 812 and the known outcomes 814 related to the machine learning model to be trained. The stage inputs 812 are from any applicable source, including text, visual representations, data, values, video representations, comparisons, and stage outputs, e.g., one or more outputs from one or more steps from FIG. 3. The known outcomes 814 are included for the machine learning models generated based on supervised or semi-supervised training or can be based on known labels, such as review classification labels. An unsupervised machine learning model is not trained using the known outcomes 814. The known outcomes 814 include known or desired outputs for future inputs similar to or in the same category as the stage inputs 812 that do not have corresponding known outputs.

The training data 810 and a training algorithm 820, e.g., one or more of the modules implemented using the machine learning model or used to train the machine learning model, are provided to a training component 830 that applies the training data 810 to the training algorithm 820 to generate the machine learning model. According to an implementation, the training component 830 is provided with comparison results 816 that compare a previous output of the corresponding machine learning model to apply the previous result to re-train the machine learning model. The comparison results 816 are used by the training component 830 to update the corresponding machine learning model. The training algorithm 820 utilizes machine learning networks or models including, but not limited to, deep learning networks such as Deep Neural Networks (DNN), Convolutional Neural Networks (CNN), Fully Convolutional Networks (FCN), and Recurrent Neural Networks (RCN), probabilistic models such as Bayesian Networks and Graphical Models, classifiers such as K-Nearest Neighbors, or discriminative models such as Decision Forests and maximum margin methods, the model specifically discussed herein, or the like.

The machine learning models used herein are trained or used by adjusting one or more weights or one or more layers of the machine learning model. For example, during training, a given weight is adjusted (e.g., increased, decreased, removed) based on training data or input data. Similarly, a layer is updated, added, or removed based on training data or input data. The resulting outputs are adjusted based on the adjusted weights or layers.

The initial training of the machine learning models may be completed by utilizing data that has been tagged. In some embodiments, this tagged data serves as an input for supervised or semi-supervised learning approaches. The tagging process can be done manually or automatically, depending on the desired level of accuracy and available resources.

Manual tagging involves human annotators who examine training data and assign appropriate classification labels based on the content and context of the training data. This method can yield high-quality labeled data, as humans can understand nuances and contextual information better than automated algorithms. However, manual tagging can be time-consuming and labor-intensive, especially when dealing with large datasets.

Automatic tagging, on the other hand, involves using algorithms, such as natural language processing techniques or pre-trained machine learning models, to assign classification labels to reviews. This approach is faster and more scalable than manual tagging but may not be as accurate, particularly when dealing with complex or ambiguous items. To improve the accuracy of automatic tagging, it can be combined with manual tagging in a semi-supervised learning approach, where a smaller set of manually tagged data is used to guide the automatic tagging process.

The data collection process can be done manually or using web-scraping techniques. Manual data collection can be time-consuming and may not cover all the available data sources. Web-scraping techniques, on the other hand, use automated tools and scripts to extract data from various sources, making the process faster and more comprehensive.

Once data has been collected and tagged with appropriate classification labels, it can be used as input for the machine learning model's training process. The model will learn to recognize patterns and features in the data that correspond to specific contexts for data. With sufficient training and accurate labeled data, the machine learning model can become adept at identifying context-specific outputs, enabling an efficient and effective model.

As one skilled in the art will appreciate in light of this disclosure, certain embodiments may be capable of achieving certain advantages, including some or all of the following: (1) improving the functionality of a computing system through a more streamlined communication interface for interacting with a display device; (2) improving the user experience in interacting with a computer system by providing the streamlined communication interface receiving dynamic and interactive information; and (3) improving the reliability of information in a database by using machine learning techniques to personalize a user experience.

It should be understood that embodiments in this disclosure are exemplary only, and that other embodiments may include various combinations of features from other embodiments, as well as additional or fewer features.

In general, any process or operation discussed in this disclosure that is understood to be computer-implementable may be performed by one or more processors of a computer system, such any of the systems or devices in the environment 100 of FIG. 1, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

A computer system, such as a system or device implementing a process or operation in the examples above, may include one or more computing devices, such as one or more of the systems or devices in FIG. 1. One or more processors of a computer system may be included in a single computing device or distributed among a plurality of computing devices to perform a computer-implemented method. A memory of the computer system may include the respective memory of each computing device of the plurality of computing devices.

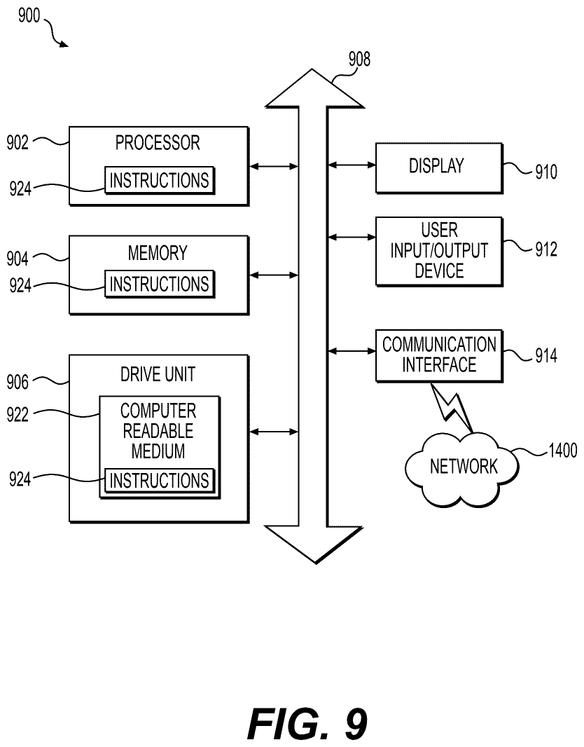

FIG. 9 is a simplified functional block diagram of a computer 900 that may be configured as a device for executing the methods described herein according to exemplary embodiments of the present disclosure. In various embodiments, any of the systems herein may be a computer 900 including, for example, a data communication interface 128 for packet data communication. The computer 900 also may include a central processing unit (“CPU”) 902, in the form of one or more processors, for executing program instructions. The computer 900 may include an internal communication bus 908, and a storage unit 906 (such as ROM, HDD, SDD, etc.) that may store data on a computer readable medium 922, although the computer 900 may receive programming and data via network communications (e.g., via network 140). The computer 900 may also have a memory 904 (such as RAM) storing instructions 924 for executing techniques presented herein, although the instructions 924 may be stored temporarily or permanently within other modules of computer 900 (e.g., processor 902 or computer readable medium 922). The computer 900 also may include input and output ports 912 or a display 910 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “items” or “articles of manufacture” typically in the form of executable code or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Aspects disclosed herein may be implemented during a robotic medical procedure where a robotic device, such as a surgical robot, a robotic tool manipulated or held by the surgeon and/or surgical robot, or other devices configured for automation perform at least a portion of a surgical procedure, such as a joint replacement procedure involving installation of an implant. Robotic device refers to surgical robot systems and/or robotic tool systems, and is not limited to a mobile or movable surgical robot. For example, robotic device may refer to a handheld robotic cutting tool, jig, burr, etc.

Any of the data collected using a humanoid robot described herein may be incorporated into any of the algorithms and/or other processes described herein, and may be used to optimize algorithms using data collected from a humanoid robot with a variety of different settings, such as range of motion, height, flexibility, and/or any other parameter described herein. Any parameter related to the humanoid robot or the medical devices may be considered associated with the medical device (e.g. prosthetic element).

Aspects disclosed herein are not limited to specific scores, thresholds, etc. that are described. For example, outputs and/or scores disclosed herein may include other types of scores such as Hoos Koos, SF-12, SF-36, Harris Hip Score, etc.

Aspects disclosed herein are not limited to specific types of surgeries and may be applied in the context of osteotomy procedures, computer navigated surgery, neurological surgery, spine surgery, otolaryngology surgery, orthopedic surgery, general surgery, urologic surgery, ophthalmologic surgery, obstetric and gynecologic surgery, plastic surgery, valve replacement surgery, endoscopic surgery, and/or laparoscopic surgery.

Aspects disclosed herein may improve or optimize surgery outcomes.

Aspects disclosed herein may augment the continuum of care to optimize post-operative outcomes for a patient. Aspects disclosed herein may recognize or determine previously unknown relationships, such as how injuries or movement at one joint affects movement at a different joint, to help optimize care, such as placement, type, and/or design of a prosthetic.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

What is claimed is:

1. A method for gathering test data for one or more medical devices, the method comprising:
programming a humanoid robot to perform a task, wherein the one or more medical devices are coupled to the humanoid robot;
receiving test data associated with the one or more medical devices during a performance of the task by the humanoid robot.

2. The method of claim 1, wherein the humanoid robot includes a phenotype selection module, the method comprising:
determining a phenotype of the humanoid robot based on the phenotype selection module.

3. The method of claim 2, wherein the phenotype selection module includes a soft tissue model, and the humanoid robot includes one or more electromechanical systems, the method comprising:
actuating the one or more electromechanical systems to conform to the phenotype determined by the phenotype selection module.

4. The method of claim 3, wherein the soft tissue model is a trained machine learning model.

5. The method of claim 2, wherein the phenotype selection module includes a task selection model configured to receive user data including a patient activity profile, and programming the humanoid robot to perform the task is based on output from the task selection model.

6. The method of claim 5, wherein the task selection model is a trained machine learning model.

7. The method of claim 2, further comprising:
receiving phenotype data from one or more data sources, the one or more data sources including prior patient data with at least one attribute in common with a current patient; and
determining the phenotype of the humanoid robot based on the phenotype data.

8. The method of claim 7, wherein the one or more data sources includes at least one of: preoperative data relating to one or more patient, postoperative data relating to one or more patients, and implant test data.

9. The method of claim 1, further comprising:
determining test outputs based on the test data, the test outputs including one or more of: durability of the one or more medical devices; performance estimates of the one or more medical devices; and postoperative activity recommendations.

10. The method of claim 1, wherein the test data is received from one or more sensors; the one or more sensors including at least one of: a force sensor, a displacement sensor, a pressure sensor, and an imaging device.

11. A system for gathering test data for one or more medical devices, the system comprising:
a humanoid robot comprising one or more processors;
one or more medical devices coupled to the humanoid robot; and
one or more sensors configured to gather test data associated with the one or more medical devices during a performance of a programmed task by the humanoid robot.

12. The system of claim 11, wherein the humanoid robot includes a phenotype selection module configured to determine a phenotype of the humanoid robot.

13. The system of claim 12, wherein the phenotype selection module includes a soft tissue model, and the humanoid robot includes one or more electromechanical systems, wherein the electromechanical systems are actuated to provide the phenotype determined by the phenotype selection module.

14. The system of claim 13, wherein the soft tissue model is a trained machine learning model.

15. The system of claim 12, wherein the phenotype selection module includes a task selection model, and programming the humanoid robot to perform the task is based on output from the task selection model.

16. The system of claim 15, wherein the task selection model is a trained machine learning model.

17. The system of claim 12, further comprising:
one or more data sources comprising phenotype data, wherein determining the phenotype of the humanoid robot is based on the phenotype data.

18. The system of claim 17, wherein the one or more data sources includes at least one of: preoperative data relating to one or more patient, postoperative data relating to one or patients, and implant test data.

19. The system of claim 11, wherein the system is further configured to determine test outputs based on the test data and to transmit the test outputs to a remote system, the test outputs including one or more of: durability of the one or more medical devices; performance estimates of the one or more medical devices; and postoperative activity recommendations.

20. A system comprising:
a programmable humanoid robot;
one or more sensors; and
one or more implants coupled to the programmable humanoid robot; wherein the programmable humanoid robot comprises:
at least one processor; and
at least one storage comprising instructions which, when executed by the at least one processor, cause the at least one processor to perform operations comprising:
receive phenotype data from one or more data sources;
determine, using a phenotype selection module, a phenotype of the humanoid robot based on the phenotype data;
adjust one or more electromechanical systems to conform the humanoid robot to the phenotype determined by the phenotype selection module;
program the humanoid robot to perform a task based on a task selection model;
receive test data associated with the one or more implants during a performance of the task by the humanoid robot; and
causing to display, on one or more display devices; test outputs based on the test data associated with the one or more implants.
